(19) United States
(12) Patent Application Publication
(10) Pub. No.: US 2018/0246111 A1
(43) Pub. Date: Aug. 30, 2018

(54) CANCER DIAGNOSTIC COMPOSITION INCLUDING ANTIBODY FOR RIBOSOMAL PROTEIN S3, CANCER DIAGNOSTIC KIT INCLUDING THE SAME, AND METHOD OF PROVIDING INFORMATION ON CANCER DIAGNOSIS USING THE SAME

(71) Applicant: Joon KIM, Seoul (KR)

(72) Inventors: Joon KIM, Seoul (KR); Hag Dong KIM, Gyeonggi-do (KR); YongJoong KIM, Seoul (KR); Yong Jun PARK, Seoul (KR)

(21) Appl. No.: 15/629,411

(22) Filed: Jun. 21, 2017

(30) Foreign Application Priority Data
Feb. 28, 2017 (KR) 10-2017-0026271
Jun. 2, 2017 (KR) 10-2017-0069289

(51) Int. Cl. G01N 33/574; C07K 16/40

(52) U.S. Cl. CPC G01N 33/57488; G01N 2333/988; C12Y 402/99018; C07K 16/40

(57) ABSTRACT

The present invention relates to a cancer diagnostic composition, a cancer diagnostic kit including the same, and a method of providing information on cancer diagnosis using the same, and more particularly, the cancer diagnostic composition includes a monoclonal antibody, a polyclonal antibody, or both monoclonal and polyclonal antibodies that specifically react with a ribosomal protein S3. The monoclonal antibody, the polyclonal antibody, or both of the monoclonal and polyclonal antibodies are useful for early diagnosis of development and metastasis of various human cancers by specifically recognizing rpS3 secreted from a cancer cell.

### CROSS-REFERENCE TO RELATED APPLICATION(S)

The present application claims priority to Korean Patent Application No. 10-2017-0026271, filed on Feb. 28, 2017 and Korean Patent Application No. 10-2017-0069289, filed on Jun. 2, 2017, which are herein incorporated by reference in its entirety.

### BACKGROUND OF THE INVENTION

### 1. Field of the Invention

The present invention relates to a cancer diagnostic composition including an antibody for a ribosomal protein s3, a cancer diagnostic kit including the same, and a method of providing information on cancer diagnosis using the same. Particularly, in order to prepare monoclonal and polyclonal antibodies, a recombinant protein, which is obtained by separating and purifying rpS3 consisting of 243 amino acids from Escherichia coli, is used as an antigen.

### 2. Description of the Related Art

A ribosomal protein small subunit 3 (rpS3) is a protein constituting ribosome, and it is also known as an enzyme which recovers damaged DNA (Kim et al., 1995 J. Biol. Chem. 270(23) 13620-9). The rpS3 is known to take various roles in cancer cell metastasis, apoptosis, and transcription in an immune response.

Recently, it is known that the various roles of the rpS3 can be regulated through modifications such as phosphorylation, methylation, monoubiquitination, and glycosylation.

The rpS3 is identified as one type of a subunit of an NF-B complex which is known as a main gene regulator of the immune response (Wan et al., 2007 Cell 131 (5), 927-39), and it is reported that the rpS3 induces the transcription of a specific gene by moving to a nucleus when a Ser209 residue is phosphorylated by IKK. It is known that the phosphorylation on the Ser209 residue is inhibited by NleHl upon infection of Escherichia coli O157:H7, thereby regulating gene expression due to NF-B (Xiaofei et al., 2009 PLoS Pathog. 5(12) e100070).

Phosphorylation of a Thr221 residue of the rpS3 may be induced by two types of kinases. One that induces the phosphorylation is PKC activated by oxidative DNA damage such as hydrogen peroxide. In this case, the phosphorylation of the Thr221 residue contributes to DNA repair by increasing the migration of rpS3 protein to the nucleus (Kim et al., 2009 BBA-Mol. Cell Res. 1793 (2), 395-405). In addition, when the phosphorylation of the Thr221 residue of the rpS3 is induced by CK2 which is activated by ionizing radiation (IR), it is reported that binding to TRAF2 is increased, thereby regulating the radioresistance of a non-small cell lung cancer (NSCLC) (Yang et al., 2013 J. Biol. Chem. 288, 2965-2975).

Akt induces the phosphorylation of the Thr70 residue of the rpS3, which is known to increase the function of rpS3 as a DNA repair enzyme in a neuronal cell line, thereby urging apoptosis (Lee et al., 2010 J. Biol. Chem. 285(38), 29457-68). Methylation of rpS3 is achieved by a protein arginine methyltransferase 1 (PRMT1) enzyme at Arg64, Arg65 and Arg67 residues, which is involved in the migration of rpS3 to a nucleolus, and assembly into the ribosome is not achieved in the absence of methylation (Shin et al., 2009 Biochem Biophys Res Commun. 429(1-2), 57-62).

Mono-ubiquitination of rpS3 occurs at a Lys214 residue, and this modification exerts an important influence on protein biosynthesis in the ribosome. The mono-ubiquitination of rpS3 protein is induced by the interference of an elongation step during a protein translation process, which acts as a signal for inducing biosynthesis of an erroneous protein, resulting in damage to a cell. The accumulation of erroneous proteins in the cell may cause various diseases related to cellular senescence (Higgins et al., 2015 Mol. Cell. 59(1), 35-49).

### SUMMARY OF THE INVENTION

Although the exact mechanism of rpS3 is unknown, N-linked glycosylation occurs at an Asn165 residue in cancer cells, in which the rpS3 is secreted out of the cells via a typical ER-Golgi secretion pathway of the cells.

The inventors of the present invention recently have studied and found that the rpS3 is rarely secreted in a normal cell, whereas large amounts of rpS3 are secreted in malignant tumor cells where metastasis frequently occurs.

An object of the present invention is to provide a cancer diagnostic composition capable of diagnosing the possibility of cancer development or cancer metastasis.

In addition, an object of the present invention is to provide a cancer diagnostic kit capable of diagnosing the possibility of cancer development or cancer metastasis.

In addition, an object of the present invention is to provide a method of providing information on cancer diagnosis, capable of diagnosing the possibility of cancer development or cancer metastasis.

Based on the above-mentioned points, the inventors of the present invention have created the present invention, in which monoclonal and polyclonal antibodies that specifically recognize the entire amino acid sequence of rpS3 are prepared, and the antibodies specifically recognize the rpS3 secreted into cell culture fluids and blood in a human body, so that metastasis and malignant states of the cancer cell are detected through a sandwich enzyme-linked immunosorbent assay (ELISA) using two kinds of antibodies, which allows early diagnosis of cancer development and cancer metastasis very accurately.

According to the present invention, there are provided a cancer diagnostic composition including monoclonal and polyclonal antibodies that specifically recognize an rpS3 secreted into blood in a human body through glycosylation of the rpS3 generated during cancer development and cancer metastasis, and a kit including the cancer diagnostic composition.

The cancer diagnostic composition includes an antibody that specifically binds to the rpS3. The antibody may be prepared from a full-length 6×His-rpS3 fusion protein labeled with a 6×His-tag.

The rpS3 has a strong basicity with an intrinsically unstructured tertiary structure, so that a precipitation phenomenon is very severe upon genetic recombination expression in bacteria. Although the rpS3 is generally fused with relatively large labeled proteins such as GST, since the labeled proteins such as GST themselves are large in size, the labeled proteins are unsuitable as an antigen for preparing an antibody required in the present invention.

Although a scheme of degrading only a GST label through enzymatic treatment may be taken into consideration, an antigen purification process becomes complicated, and it is difficult to acquire a high concentration level required for antigen immunization.

The inventors of the present invention have found that the 6×His-tag consisting of only 6 amino acids is more economical because it has a relatively small size compared to the GST, and it is more efficient to purify high-concentration antigen proteins in large quantities by dissolving the rpS3, which is aggregated under a denaturing condition, with guanidine hydrochloride.

Using the full-length 6×His-rpS3 fusion protein as an antigen may increase the efficiency of preparing an antibody specifically binding to the rpS3.

The above antibody may be a monoclonal antibody or a polyclonal antibody.

The monoclonal antibody may be prepared by using a phage antibody library technique (Clackson et al., Nature, 352:624-628, 1991; Marks et al., J. Mol. Biol., 222:58, 1-597, 1991), or a hybridoma scheme (Kohler and Milstein (1976) European Jounral of Immunology 6:511-519), which is generally known in the art.

The polyclonal antibody may be prepared by a scheme generally known in the art, in which the rpS3 protein antigen is injected into an animal, and blood of the animal is collected to acquire a serum including the antibody.

Such a polyclonal antibody may be prepared from any animal host species such as goats, rabbits, sheep, monkeys, horses, pigs, mice, rats, cows, and dogs.

The antibody prepared by the above scheme may be separated and purified by using schemes such as gel electrophoresis, dialysis, salt precipitation, ion-exchange chromatography, and affinity chromatography.

The antibody includes a complete shape having two full-length light chains and two full-length heavy chains, as well as a functional fragment of an antibody molecule. The functional fragment of the antibody molecule refers to a fragment having at least an antigen binding function, and includes Fab, F(ab′), F(ab′)2, and Fv.

A cancer diagnostic kit includes the cancer diagnostic composition. The cancer diagnostic kit may further include a secondary antibody to increase the accuracy of measuring a secretion level or a secretion amount of the rpS3.

An analytical scheme for measuring an amount of protein includes immunoblot (western blot), enzyme linked immunosorbent assays (ELISA), radioimmunoassays, radioimmunodiffusion, ouchterlony immunodiffusion, rocket immunoelectrophoresis, tissue immunostaining, immunoprecipitation assays, complement fixation assays, FACS, protein chips and the like, but the analytical scheme is not limited thereto.

A sandwich enzyme-linked immunosorbent assay (sandwich ELISA) may be used to measure the secretion level or the secretion amount of the rpS3. The sandwich enzyme-linked immunosorbent assay may detect metastasis and malignant states of the cancer cell by using two kinds of antibodies, which allows early diagnosis of cancer development and cancer metastasis very accurately.

Since the above-described cancer diagnostic composition has been described in detail previously, a detailed description thereof will be omitted.

A method of providing information on cancer diagnosis includes: measuring a secretion level or a secretion amount of rpS3 secreted from a cancer cell in blood or a cell culture fluid by using an antibody specifically binding to the rpS3; and comparing a result of the measurement with a secretion level or a secretion amount of rpS3 secreted from a normal cell.

For example, the method of providing the information on the cancer diagnosis may include: (a) measuring an expression level of rpS3 from a biological specimen of a normal person and a biological specimen of a detection target organism; and (b) determining that the expression level of rpS3 secreted from the biological specimen of the detection target organism has possibility of cancer development or cancer metastasis, if the expression level of rpS3 secreted from the biological specimen of the detection target organism is increased compared to the expression level of rpS3 measured from the biological specimen of the normal person.

A sandwich enzyme-linked immunosorbent assay (sandwich ELISA) may be used to measure the secretion level or the secretion amount of the rpS3. The sandwich enzyme-linked immunosorbent assay may detect metastasis and malignant states of the cancer cell by using two kinds of antibodies, which allows early diagnosis of cancer development and cancer metastasis very accurately.

The specimen may include tissue, cells, blood, serum, plasma, saliva, cerebrospinal fluid, sweat, urine, ascite fluid, and peritoneal fluid, but is not limited thereto. The specimen is preferred to be blood, serum, or plasma.

A diagnosis target cancer may include fibrosarcoma, plasmacytoma, bronchial cancer, leukemia, breast cancer, ovarian cancer, uterine cancer, skin cancer, bladder cancer, prostate cancer, renal cancer, thyroid cancer, esophageal cancer, gastric cancer, liver cancer, colon cancer, pancreatic cancer, lung cancer, brain tumor, osteosarcoma, malignant angioma, and malignant lymphoma.

According to the present invention, the level of rpS3 secreted from the cancer cell can be accurately and easily measured.

According to the present invention, the possibility of cancer development or cancer metastasis can be diagnosed in an early stage.

According to the present invention, the level of rpS3 protein secreted from the cancer cell can be accurately and easily measured by using monoclonal and polyclonal rpS3 antibodies, thereby allowing the early diagnosis of the development and the metastasis of various cancers.

### BRIEF DESCRIPTION OF THE DRAWINGS

FIG. 1 is a plasmid genetic map showing expression of an entire protein coding gene of a human rpS3, for use as an immunizing antigen.

FIG. 2 is a plasmid genetic map showing expression of an entire protein coding gene of a human rpS3 to express GST-rpS3 that fixes a prepared monoclonal antibody and a prepared polyclonal antibody to Affi-Gel 10 resin, for affinity pure separation and purification.

FIG. 3 shows western blotting of the human rpS3 by using the monoclonal and polyclonal antibodies.

FIG. 4A is an image showing that the rpS3 is secreted more in a malignant cancer than in a control group. In fibrillarin and other ribosomal proteins used as the control group, this phenomenon is not shown.

FIG. 4B is an image showing that the rpS3 is secreted more in a blood cancer and a fetal fibroblast than in a normal cell.

FIG. 5A shows that a secretion rate of rpS3 in serum of a cancer patient is higher than that of a normal person, as a result of comparing the secretion of rpS3 protein in the serum of the normal person with the secretion of rpS3 protein in the serum of patients having a gastric cancer, a liver cancer, and a colon cancer. Cell lysate is a lane that confirms the presence or absence of the target protein by breaking down the cell.

FIG. 5B shows a result of performing the western blotting after performing immune-precipitation (IP) which uses a self-prepared polyclonal antibody by using serum of a gastric cancer patient. In other words, the western blotting is performed after the immune-precipitation using the polyclonal antibody prepared by the research team of the present invention is performed using the serum collected from the cancer patient. As a result, although rpS3 band confirmed on the cell lysate used as a control is not shown in globulin G (denoted as R IgG) used as an immune-precipitation control, the rpS3 band is confirmed on an experiment (denoted as rpS3) where the polyclonal antibody is added.

This indicates that the polyclonal antibody prepared by the research team of the present invention can accurately detect the rpS3 contained in the serum, and the antibody prepared using a full-length rpS3 immunogen by the research team of the present invention is an excellent antibody for the immune-precipitation. In addition, it is confirmed that the sandwich ELISA can be successfully performed through this scheme.

FIG. 6A shows a result of the sandwich ELISA performed by using 1 microgram (μg) of primary monoclonal antibody and 7 microgram (μg) of secondary polyclonal antibody. Only rpS3 antigen of 1000 pg/ml is specifically detected.

FIG. 6B shows a result of the sandwich ELISA performed by using 2.5 microgram (μg) of primary monoclonal antibody and 7 microgram (μg) of secondary polyclonal antibody. Only rpS3 antigen of 1000 pg/ml is specifically detected.

FIG. 6C shows a result of the sandwich ELISA in which rpS3 is secreted more by 2.5 times or more in MDA-MB231 malignant tumor cells than in MCF7 tumor cells.

FIG. 6D shows a result of the sandwich ELISA in which rpS3 is secreted more in tumor cells of gastric cancer patients, liver cancer patients and colon cancer patients than in normal cells.

### DETAILED DESCRIPTION OF THE INVENTION

Hereinafter, the present invention will be described in more detail with reference to examples and drawings. It will be obvious that the purpose of the following examples is to specifically describe the present invention in detail and the examples are not to be construed to limit the scope of the present invention.

### Example 1: Preparation of Antigen

In the present invention, in order to prepare an antibody capable of inducing effective immune-precipitation of rpS3, a full-length human rpS3 (ribosomal protein small subunit 3, Genbank accession number NM 001005.4) gene is inserted into a pET-21a vector and then expressed so as to be used as an immunogen.

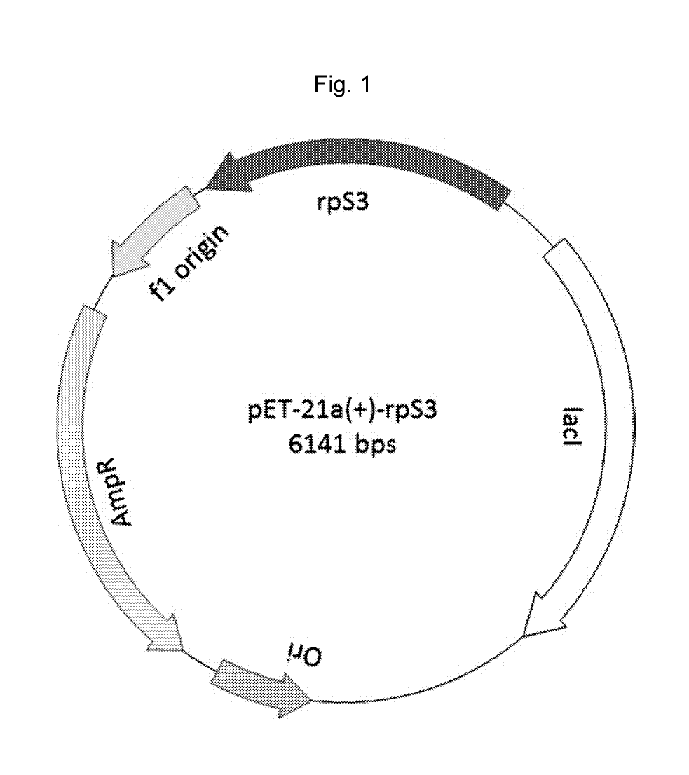

An EcoRI-XhoI restriction enzyme site among multiple cloning sites (MCS) of pET-21a (Merck Millipore-Novagen Cat. #69740) is used to design a plasmid capable of expressing the full-length rpS3 labeled with 6 histidine amino acids (6×His) by inserting the entire protein coding gene (732 bp) of a human rpS3 (see FIG. 1).

The plasmid is transformed into a bacterial strain BL-21 (Amersham Pharmacia Biotech, Cat. #27-1542-01) for expressing an E. coli-family protein, thereby preparing genetic recombinant bacteria.

The genetic recombinant bacteria are cultured in a lysogeny broth (LB) liquid culture medium at 37° C. for 3 hours, strongly induced in a culture medium where isopropyl-D-1-thiogalactopyranoside (IPTG) of 0.5 mM is added, and cultured at 30° C. for 4 hours.

A cell wall and a cell membrane of the bacteria thus cultured are degraded by using a sonicator. In order to inhibit the recombinant rpS3 from entering and being precipitated in an inclusion body by 80% thereof or more due to the properties of the recombinant rpS3, a supernatant of the recombinant rpS3 is flown into Nickel-NTA agarose resin with strong properties of binding to 6×His-tags, under a denaturing condition in a buffer solution containing guanidine chloride which has concentration of 6M, so as to allow the 6×His-labeled rpS3 to bind to agar resin through a nickel ion.

The rpS3-bound resin is washed with a washing solution of 20 times the volume of the resin to remove unspecific proteins, and the fused protein is collected from the resin by injecting an elution buffer where imidazole is added. The resulting solution is dialysis-purified through a cellulose semipermeable membrane to remove imidazole and guanidine, and the final 6×His-rpS3 fusion protein has a concentration of 6.01 mg/mL.

### Example 2: Immunization

In order to prepare the monoclonal antibody, the 6×His-rpS3 fusion protein obtained in Example 1 is mixed with oil adjuvant, and the mixed solution is injected intravenously into a BALB/c mouse. The mouse is subjected to secondary immunization again two weeks later.

In order to prepare the polyclonal antibody, the 6×His-rpS3 fusion protein obtained in Example 1 is injected intravenously into a rabbit of about 20 weeks old. The rabbit is subjected to secondary inoculation two weeks after the first inoculation, and subjected to tertiary inoculation three weeks thereafter to improve the immunization.

### Example 3

Preparation and Selection of Monoclonal Antibody Hybridoma

After the second immunization, the mouse is slaughtered in a humane manner according to the Animal Welfare Act to collect a spleen. Then, B lymphocytes are separated, and the B lymphocytes are fused with a SP2/0 cell, which is a myeloma cell line, by using an electric pulse fusion device.

In order to separate the fused cells from unfused cells, the cells are cultured for 3 weeks or longer in an HAT culture medium where hypoxanthin, aminopterin and thymidine are added, and the unfused cells are removed while the fused cells are obtained.

These hybridoma are separated into pure monoclonal groups through limiting dilution, and it is analyzed by enzyme-linked immunosorbent assay (ELISA) whether antibodies are secreted specifically binding to rpS3 in each group, thereby obtaining hybridoma producing the rpS3 monoclonal antibody.

This cell permanently secretes a monoclonal antibody that specifically reacts with rpS3, and the monoclonal antibody can be obtained by collecting the supernatant which cultured the cell.

Purification of Antibodies

The following process is performed to purify the polyclonal antibody in the plasma of the blood collected from the rabbit in a humane manner after the third immunization in Example 2.

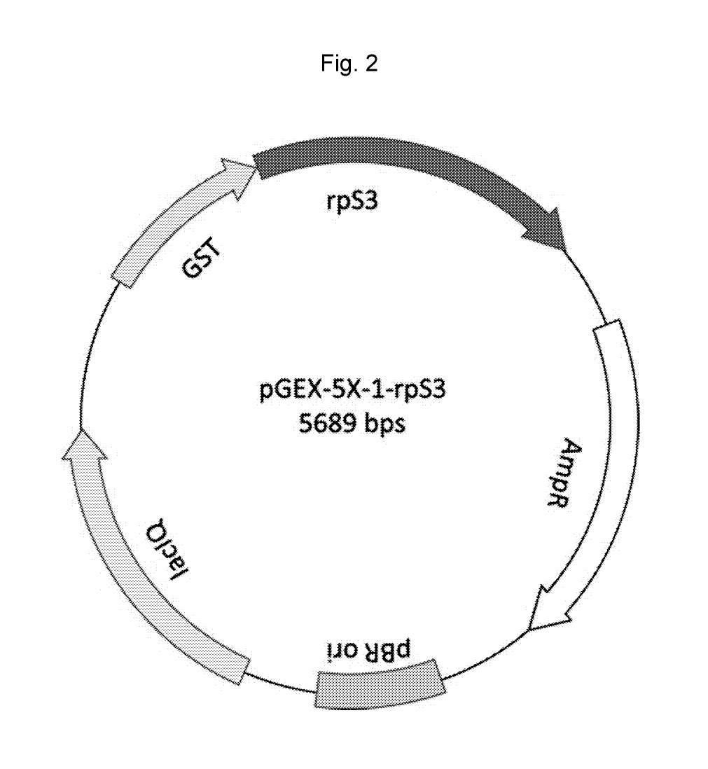

The coding gene of rpS3 is fused by using EcoRI and SalI among MCS of the pGEX-5X-1 (Amersham Pharmacia Biotech Cat. #27-4584-01) vector, and a plasmid for expressing GST-rpS3 fusion recombinant protein fused with GST protein is prepared (see FIG. 2).

The plasmid is transformed at a BL-21 bacterium strain, and the GST-rpS3 recombinant protein is expressed in the same manner as in Example 1. Thereafter, a cell solution is flown into GST-sepharose 4B resin (GE Healthcare Life Sciences, Cat. 17075601) to collect the GST-rpS3, and the GST-rpS3 is purified.

The GST-rpS3 purified by the above scheme is reacted with Affi-Gel 10 resin (Bio-Rad, Cat. #1536099) to prepare a GST-rpS3 immobilized resin, the plasma of blood collected from the immunized rabbit is passed through the prepared resin, and an elution buffer (100 mM Glycine-HCl pH2.4, 150 mM NaClm 4° C.) is reacted for 2 minutes, thereby separating only antibodies specifically binding to rpS3.

### Experimental Example 1

Western Blotting Test Using Monoclonal Antibody and Polyclonal Antibody

Whether the hybridoma secreting the monoclonal antibody obtained in the above example can actually detect the rpS3 present in human cells or not is confirmed by the western blotting.

HeLa cells, which is a representative human uterine cancer cell line derived from a human, are inserted into a Tris-NaCl-NP40 buffer (TNN buffer) containing protease inhibitors, and dissolved through a freeze-thawing scheme.

The cell lysate obtained through the above process is quantified to prepare total cell protein of 20 μg, an electrophoresis is performed in 10% SDS acrylamide gel, and the cell lysate is moved to a nitrocellulose membrane.

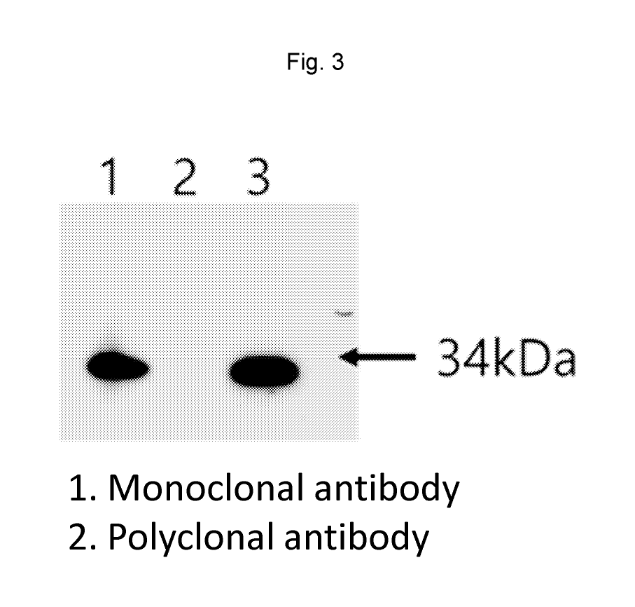

The total cell protein rooted on the nitrocellulose membrane was subject to a blocking reaction for one hour using a blocking solution formed of dried skim milk, and reacted with a culture solution (FIG. 3-1 lane) containing the antibody secreted from monoclonal antibody hybridoma and a solution, in which the polyclonal antibody (FIG. 3-3 lane) using the rabbit is diluted at a concentration of 0.2 μg/mL, for one hour at the room temperature.

Those samples are treated with horseradish peroxidase (HRP) conjugated goat anti-rabbit and anti-mouse IgG serum for one hour at the room temperature, treated with chemiluminescence substrate (Roche Diagnostics cat. #1 501 399), and sensitized to an X-ray film (see FIG. 1).

According to the experimental result, it is confirmed that the monoclonal antibody (Lane 1) and the polyclonal antibody (Lane 3) prepared in the present invention can specifically detect only rpS3 among total proteins obtained from HeLa cells.

### Experimental Example 2

Research on Secretion Rate of rps3 in Culture Medium of Cancer Cell Line Using Antibody

The polyclonal rpS3 antibody produced in Example 3 is used to identify rpS3 secreted in a culture medium by using MDA-MB-231, which is a relatively malignant breast cancer cell line, and a MCF7 cell line as a control group of MDA-MB-231.

The cell lines are cultured, and then re-cultured in a serum-free culture medium for 16 hours. The culture medium is collected and centrifuged at 1,000 rpm for 3 minutes to screen dead cells in the culture medium.

After removing the pellet formed on the bottom of the container, the culture medium is collected in an amicon tube and condensed to 1/200 by using a centrifuge. The condensed culture medium is developed on 12% SDS-PAGE and transferred to a polyvinylidene difluoride membrane (PVDF membrane), the membrane is reacted with a blocking solution for 1 hour, antibodies of ribosomal proteins S3, RACK1, L11, L13 and fibrillarin, which is a protein present in an intracellular nucleus, are applied so as to perform the western blot.

Coomassie brilliant blue (CBB) staining is performed to confirm that amounts of proteins used in the western blot between the two cell lines are the same.

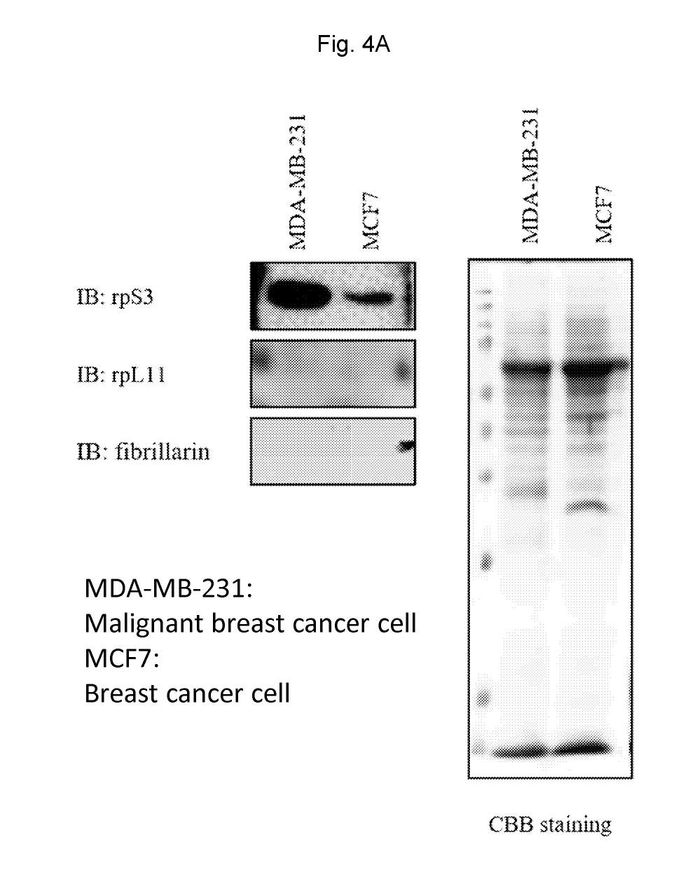

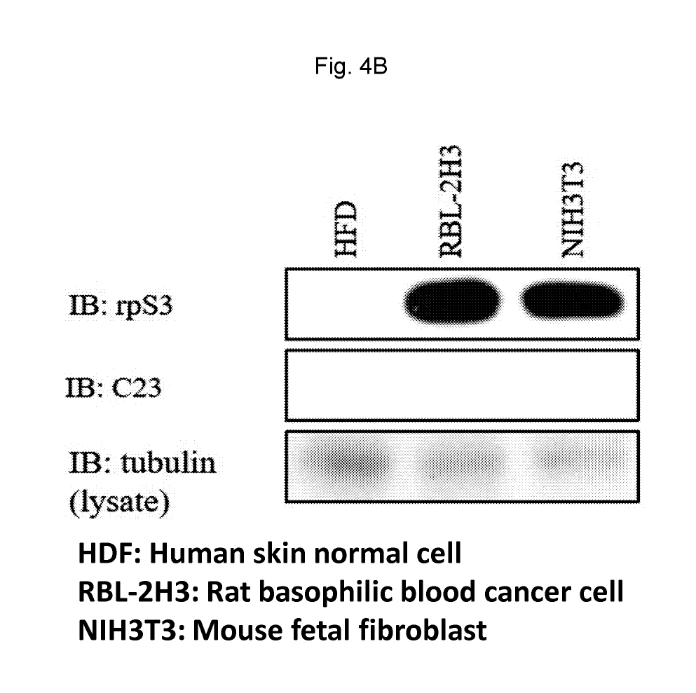

The secretion rate measurement western scheme is performed under a condition that the same amount of protein is used in the CBB staining. The polyclonal rpS3 antibody prepared in Example 3 is used to confirm that rpS3 is secreted more extracellularly from the MDA-MB-231 breast cancer cell line, which is a relatively more malignant tumor cell, than from the MCF7 breast cancer cell line (see FIG. 4A).

In addition, the fibrillarin protein is an intracellular protein that has not been detected in the above measurement scheme, which indicates that no proteins are released by dead cells during the process of the experiment. Therefore, the measured rpS3 is proved to be a protein released out of the cell through the secretion process.

It is confirmed that the secretion rate of RTL-2H3 (Rattus basophilic leukemia: rat basophil), which is a relatively malignant tumor cell, is significantly increased compared with a human dermal fibroblast (HDF) in a normal cell line (see FIG. 4B).

### Experimental Example 3

Research on Rps3 Secretion Rate in Blood of Gastric Cancer Patient and Normal Person by Using Polyclonal Antibody and Performing Immune-Precipitation (IP) Scheme

On the basis of the facts verified in Experimental Example 2, in vivo experiments are carried out on blood of a gastric cancer patient, a liver cancer patient, and a colon cancer (sigmoid-colon cancer) patient, and blood of a normal person.

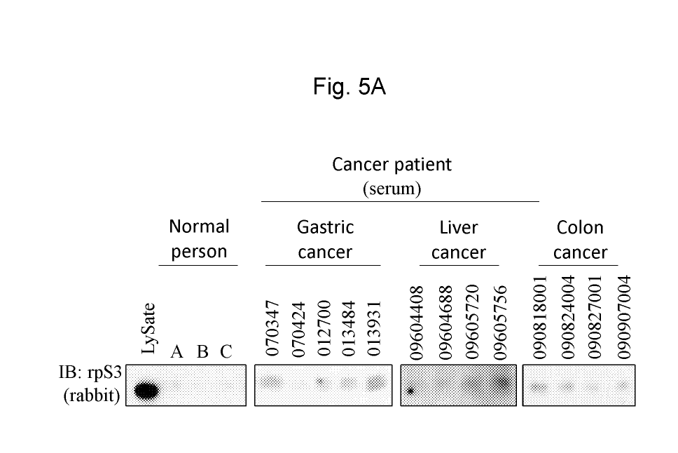

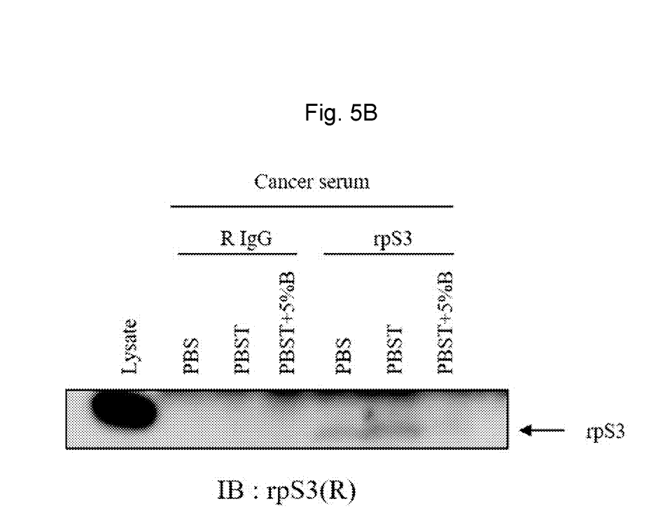

Only the serum is filtered through centrifugation of 3,000 rpm/10 min/4° C. from the donated blood, and the collected serum is diluted the ratio of 1:20 in a phosphate buffered saline (PBS) solution containing protease inhibitor cocktail to develop SDS-PAGE, in which the ribosomal protein S3 is identified by the western blotting (see FIG. 5A). The polyclonal S3 antibody prepared in Example 3 is used to identify the S3 protein. Cell lysate is a lane that confirms the presence or absence of the target protein by breaking down the cell.

The immune-precipitation is performed by using the polyclonal S3 antibody in the serum separated from the blood collected from the gastric cancer patient.

A phosphate-buffered saline solution (PBS of FIG. 5B), PBS containing 0.1% tween20 (PBST of FIG. 5b), and PBST containing 5% skim milk (PBST+5% B of FIG. 5b), which are 900 μl in total, are mixed with the collected serum of 100 μl, reacted with 2 μg of polyclonal antibody for 2 hours, and then reacted with agarose bead for 2 hours to extract protein reacted with the antibody.

The resulting solution is developed on 10% SDS-PAGE and transferred to a polyvinylidene difluoride membrane (PVDF membrane), and the western blot is performed using the polyclonal antibody.

As a result, although rpS3 band confirmed on the cell lysate used as a control group is not shown in globulin G (denoted as R IgG) used as an immune-precipitation control group, the rpS3 band is confirmed on an experiment (denoted as rpS3) where the polyclonal antibody is added.

Ultimately, through the western blot and immune-precipitation schemes, it is confirmed through the experiment that polyclonal antibodies can accurately detect rpS3 contained in serum, and can be effectively used in a sandwich ELISA.

### Experimental Example 4

Performing Sandwich Enzyme-Linked Immunosorbent Assay (ELISA) Using Monoclonal and Polyclonal Antibodies (Using Culture Medium of Cancer Cell Lines)

The recombinant rpS3 synthesized by the method described in Example 1 and the ribosomal protein small subunit 5 (rpS5) ribosomal protein, which is the control group, are used to establish the ELISA scheme by using the monoclonal antibody and the polyclonal antibody prepared in Example 3.

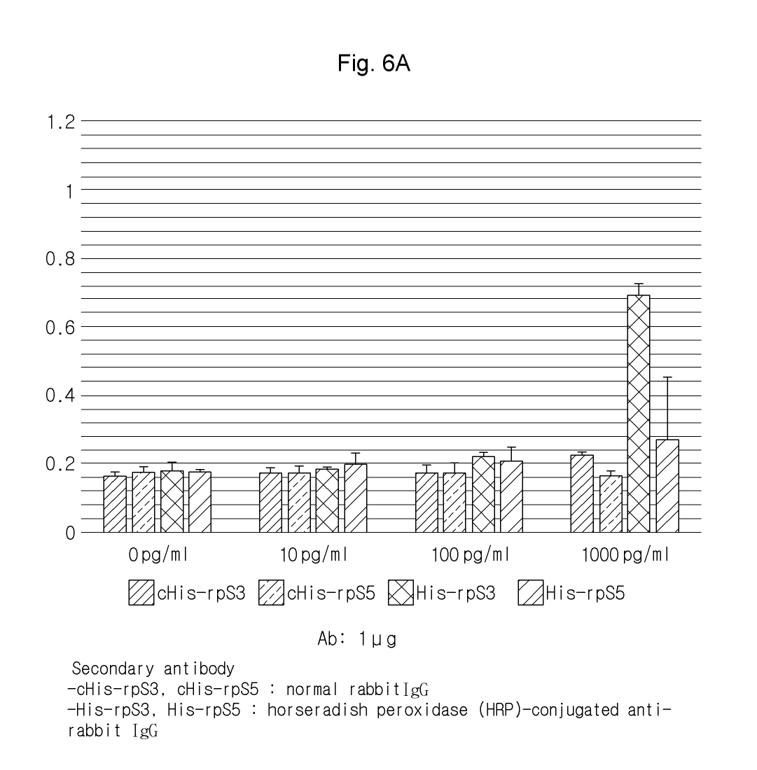

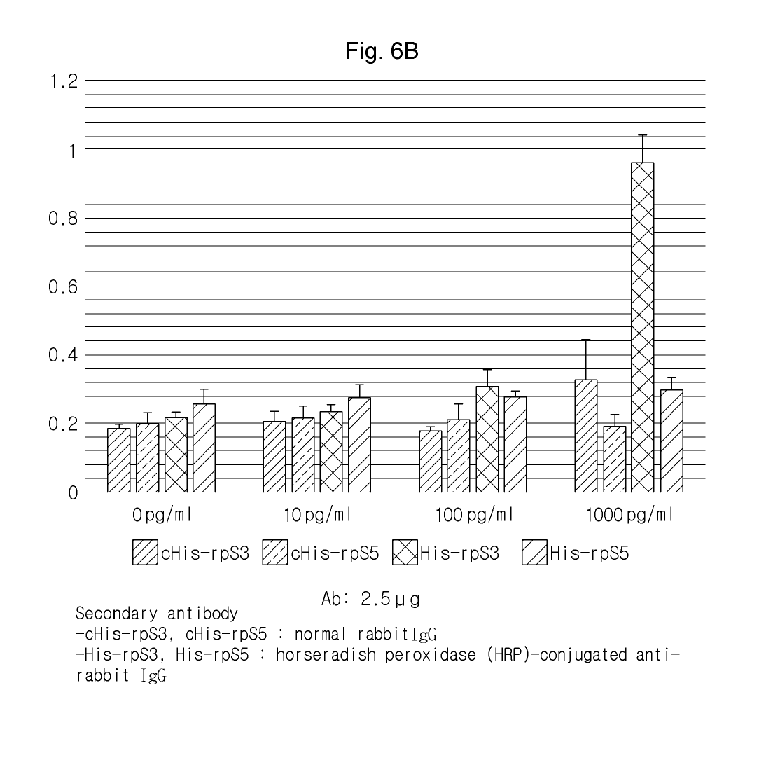

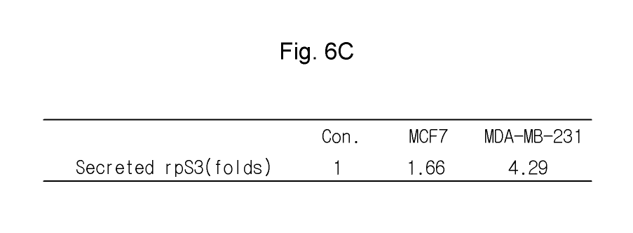

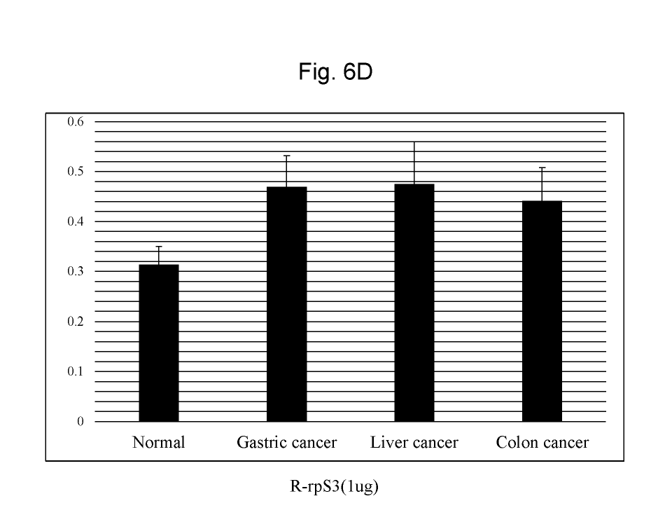

As shown in FIGS. 6a and 6b, a preliminary experiment is conducted.

First, a 96-well plate is coated with the monoclonal rpS3 antibody, washed 5 times with the phosphate-buffered saline (PBS) solution, and incubated for 16 hours in PBS mixed with 1% bovine serum albumin (BSA).

Recombinant proteins are injected into wells, respectively, reacted at room temperature for 2 hours, and washed 5 times with the PBS solution. Then, the recombinant proteins are reacted using the polyclonal antibody prepared in Example 3 at room temperature for 1 hour, and finally washed 5 times with the PBS solution.

After the reaction, the specimen is reacted again with horseradish peroxidase (HRP)-conjugated anti-rabbit IgG as a secondary antibody and washed 5 times. In addition, the TMB solution is added as a substrate for the secondary antibody, and after 30 minutes, the STOP solution is added and the absorbance at wavelength of 450 nm is measured in a microplate reader (see FIGS. 6A and 6B).

As a result, it is confirmed that the monoclonal antibody prepared in Example 3 can detect the target protein sufficiently in an amount of 1 μg. Based on this, rpS3 (extracellularly secreted rpS3) on the culture medium is measured in the same manner, in which 50 μl of the culture medium where the MCF7 cell line and the MDA-MB-231 cell line are cultured is injected into each well, the absorbance at wavelength of 450 nm is measured in the microplate reader, and the measured value is plotted as the increase/decrease ratio with respect to the control group (see FIG. 6C).

As a result, an increase of 1.66 times is observed in the MCF7 cell line, which is a human breast cancer cell, and a remarkable increase of 4 times or more is observed in the MDA-MB-231 cell line, which is a malignant human breast cancer cell. In addition, this result shows that the amount of rpS3 secreted outward of the cell in the culture medium of the MDA-MB-231 cell line, which is known as a malignant tumor cell where metastasis occurs more frequently, is more than twice the amount of rpS3 secreted outward of the cell in the MCF7 cell line, which is a breast cancer cell line.

This indicates that the sandwich ELISA scheme using the monoclonal antibody and the polyclonal antibody prepared in Example 3 can accurately measure the degree of metastasis in various cancer cells at an early stage.

The sandwich enzyme-linked immunosorbent assay (ELISA) using the monoclonal and polyclonal antibodies are performed in the blood of gastric cancer patients, liver cancer patients, and colon cancer patients. The rpS3 is secreted more in tumor cells of gastric cancer patients, liver cancer patients, and colorectal cancer patients than in normal cells (see FIG. 6D).

The present invention has been described in detail according to the present invention, and it will be apparent to those skilled in the art that the specific description is only for a preferred embodiment and is not limited to the scope of the present invention. Accordingly, the actual scope of the present invention will be defined by the appended claims and their equivalents.

What is claimed is:

1. A cancer diagnostic composition, wherein the cancer diagnostic composition includes an antibody specifically binding to rpS3, and a level of rpS3 secreted from a cancer cell is measured.

2. The cancer diagnostic composition of claim 1, wherein the antibody is prepared by taking a full-length rpS3 labeled with a 6×His-tag as an antigen.

3. The cancer diagnostic composition of claim 1, wherein the antibody includes a monoclonal or polyclonal antibody.

4. The cancer diagnostic composition of claim 1, wherein the rpS3 is measured from a patient specimen.

5. The cancer diagnostic composition of claim 1, wherein a secretion level of the rpS3 is measured through an enzyme-linked immunosorbent assay (ELISA) scheme.

6. A cancer diagnostic kit including a cancer diagnostic composition of claim 1.

7. The cancer diagnostic kit of claim 6, further including an efficient secondary antibody capable of immune-precipitation (IP) to increase accuracy with respect to secreted rpS3.

8. A method of providing information on cancer diagnosis, the method comprising:
(a) measuring an expression level of rpS3 from a biological specimen of a normal person and a biological specimen of a detection target organism; and
(b) determining that the expression level of rpS3 secreted from the biological specimen of the detection target organism has possibility of cancer development or cancer metastasis, if the expression level of rpS3 secreted from the biological specimen of the detection target organism is increased compared to the expression level of rpS3 measured from the biological specimen of the normal person.

9. The method of claim 8, wherein the expression level of rpS3 is measured using an antibody that specifically binds to the rpS3.
